(19) United States
(12) Patent Application Publication
(10) Pub. No.: US 2026/0249034 A1
(43) Pub. Date: Aug. 27, 2026

(54) CLAMPING VALVE ASSEMBLY FOR A VENTILATOR

(71) Applicant: The Children's Hospital of Philadelphia, Philadelphia, PA (US)

(72) Inventors: Nicholas J. Widmann, Philadelphia, PA (US); M. Katie Weeks, Philadelphia, PA (US); Todd J. Kilbaugh, Philadelphia, PA (US)

(73) Assignee: The Children's Hospital of Philadelphia, Philadelphia, PA (US)

(21) Appl. No.: 18/877,413

(22) PCT Filed: Jun. 19, 2023
(86) PCT No.: PCT/US2023/025656

§ 371 Date: Dec. 20, 2024

Related U.S. Application Data
(60) Provisional application No. 63/354,108, filed on Jun. 21, 2022.

(51) Int. Cl. A61M 16/20; A61M 16/00; A61M 16/12; A61M 16/18; A61M 16/22

(52) U.S. Cl. CPC A61M 16/202; A61M 16/0066; A61M 16/0078; A61M 16/009; A61M 16/12; A61M 16/18; A61M 16/22; A61M 2205/502; A61M 2205/8206

(57) ABSTRACT

A ventilator clamping valve assembly comprising: a base with a clamp cradle having first and second cradle surfaces; a clamp movably mounted to the base and having first and second clamp surfaces; a motor to drive the clamp; a first inlet port; a first outlet port; a second inlet port; a second outlet port; a first tube extending through a first volume defined between the first clamp surface and the first cradle surface, and fluidly connecting the first inlet port and the first outlet port; and a second tube extending through a second volume defined between the second clamp surface and the second cradle surface and fluidly connecting the second inlet port and the second outlet port.

### CROSS-REFERENCE TO RELATED APPLICATION

This application claims priority to and the benefit of U.S. Provisional Application No. 63/354,108, filed on Jun. 21, 2022, which is incorporated herein by reference.

### FIELD OF THE INVENTION

The subject matter disclosed herein relates ventilator devices, including small-footprint and modular ventilators.

### BACKGROUND OF THE INVENTION

Various medical ventilators are known in the art. Such ventilators may be essentially fixed in place, or readily portable for use in different locations. While ventilators are in common use, and widely accepted, the inventors have determined that conventional ventilators, particularly portable ventilators, can be excessively bulky, expensive to manufacture, and otherwise have drawbacks that limit their utility. Thus, there is a need to advance the state of the art of medical ventilators.

### SUMMARY OF THE INVENTION

In one aspect, there is provided a ventilator clamping valve assembly comprising: a base; a clamp cradle positioned on the base and having a first cradle surface and a second cradle surface; a clamp movably mounted relative to the base, the clamp having a first clamp surface facing the first cradle surface to define a first volume between the first clamp surface and the first cradle surface, and a second clamp surface facing the second cradle surface to define a second volume between the second clamp surface and the second cradle surface; a motor operatively connected to the clamp and configured to move the clamp between a first clamp position in which the first volume has a starting first volume value and the second volume has a starting second volume value, and a second clamp position in which the first volume has a final first volume value that is different from the starting first volume value, and the second volume has a final second volume value that is different from the starting second volume value; a plurality of ports comprising: a first inlet port, a first outlet port, a second inlet port, and a second outlet port; a first tube extending through the first volume and fluidly connecting the first inlet port and the first outlet port; and a second tube extending through the second volume and fluidly connecting the second inlet port and the second outlet port.

Also provided is a ventilator system comprising: a housing; a clamping valve assembly connected to the housing and comprising: a base, a clamp cradle positioned on the base and having a first cradle surface and a second cradle surface, a clamp movably mounted relative to the base, the clamp having a first clamp surface facing the first cradle surface to define a first volume between the first clamp surface and the first cradle surface, and a second clamp surface facing the second cradle surface to define a second volume between the second clamp surface and the second cradle surface, and a motor operatively connected to the clamp and configured to move the clamp between a first clamp position in which the first volume has a starting first volume value and the second volume has a starting second volume value, and a second clamp position in which the first volume has a final first volume value that is different from the starting first volume value, and the second volume has a final second volume value that is different from the starting second volume value; a plurality of ports comprising: a first inlet port, a first outlet port, a second inlet port, and a second outlet port, a first tube extending through the first volume and fluidly connecting the first inlet port and the first outlet port, and a second tube extending through the second volume and fluidly connecting the second inlet port and the second outlet port; a blower having a blower inlet and a blower outlet, the blower outlet being fluidly connected to the first inlet port when the clamping valve assembly is connected to the housing; and a controller configured to operate the blower to generate a flow of gas into the first inlet, and to operate the motor to reciprocate the clamp between the first position and the second position.

Also provided is a method for assembling or servicing a ventilator, the method comprising: accessing an interior of a ventilator housing; removing and replacing at least one of a first inlet port, a first outlet port, a second inlet port, and a second outlet port of a clamping valve assembly attached within the interior of the ventilator housing while a remainder of the clamping valve assembly remains attached within the interior of the ventilator housing; and removing and replacing one or more of a first tube fluidly connecting the first inlet port and the first outlet port, and a second tube fluidly connecting the second inlet port and the second outlet port.

Also provided is a service unit for a ventilator, the service unit comprising: a sealed package comprising a sterile interior volume; a first inlet port located in the sterile interior volume; a first outlet port located in the sterile interior volume; a second inlet port located in the sterile interior volume; a second outlet port located in the sterile interior volume; a first tube located in the sterile interior volume; and a second tube located in the sterile interior volume.

Also provided is a method for operating a ventilator clamping valve assembly comprising a clamp cradle having a first cradle surface and a second cradle surface, a clamp having a first clamp surface facing the first cradle surface to define a first volume between the first clamp surface and the first cradle surface and a second clamp surface facing the second cradle surface to define a second volume between the second clamp surface and the second cradle surface, a first tube extending through the first volume, and a second tube extending through the second volume. The method comprises: (a) moving the first clamp surface towards the first cradle surface to simultaneously decrease the first volume to compress the first tube and increase the second volume to decompress the second tube; (b) moving the second clamp surface towards the second cradle surface to simultaneously decrease the second volume to compress the second tube and increase the first volume to decompress the first tube; and cyclically repeating steps (a) and (b) according to a respiration rate of a patient.

Also provided is a ventilator clamping valve assembly comprising: a base; a plurality of ports comprising: a first inlet port, a first outlet port, a second inlet port, and a second outlet port; a first tube connecting the first inlet port and the first outlet port; a second tube connecting the second inlet port and the second outlet port; a first valve body configured to selectively change a cross-sectional area of the first tube; a second valve body configured to selectively change a cross-sectional area of the second tube; one or more motors connected to the first valve body and the second valve body and control electronics configured to: operate the one or more motors to simultaneously move the first valve body to reduce the cross-sectional area of the first tube and move the second valve body to increase the cross-sectional area of the second tube; and operate the one or more motors to simultaneously move the first valve body to increase the cross-sectional area of the first tube and move the second valve body to decrease the cross-sectional area of the second tube.

### BRIEF DESCRIPTION OF THE FIGURES

FIG. 1 is an isometric view of an exemplary ventilator.

FIG. 2 is a top plan view of the ventilator of FIG. 1, shown with a top cover removed.

FIG. 3 is an isometric view of an exemplary blower.

FIG. 4 is an isometric view of an exemplary pipe system.

FIG. 5 is an isometric view of an exemplary electronics module.

FIG. 6 is an isometric view of an exemplary battery.

FIG. 7 is a top plan view of an exemplary clamping valve assembly.

FIG. 8 is an isometric view of the clamping valve assembly of FIG. 7.

FIG. 9 is a schematic view of exemplary clamping valve assembly showing the clamp, cradle and tubes.

FIGS. 10-13 are isometric views of the clamping valve assembly of FIG. 7 shown in various states of assembly.

FIGS. 14 and 15 schematically illustrate a clamping valve assembly with the clamp in two opposite end positions.

FIG. 16 is a schematic illustration of a ventilator system connected to a patient.

FIGS. 17-20 are data plots showing operational data of an exemplary ventilator.

FIGS. 21-24 show various exemplary ventilator systems.

FIG. 25 illustrates another exemplary embodiment of a clamping valve assembly.

FIG. 26 illustrates another exemplary embodiment of a clamping valve assembly.

FIG. 27 illustrates another exemplary embodiment of a clamping valve assembly.

### DETAILED DESCRIPTION OF THE INVENTION

In the following detailed description, numerous specific details are set forth by way of examples in order to provide a thorough understanding of the relevant teachings. However, it should be apparent to those skilled in the art that the present teachings may be practiced without such details. In other instances, well known methods, procedures, components, and circuitry have been described at a relatively high-level, without detail, in order to avoid unnecessarily obscuring aspects of the present teachings.

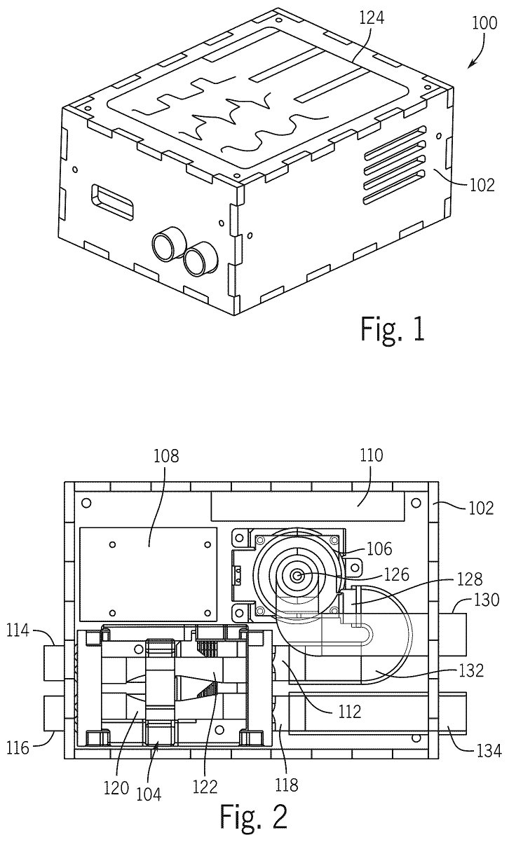

FIGS. 1 and 2 illustrate an exemplary embodiment of a ventilator 100. The ventilator 100 generally includes a housing 102 that holds a clamping valve assembly 104, a blower 106, control electronics 108, and a battery 110 and/or a power supply port (e.g., power cord or the like) (not shown).

The clamping valve assembly includes a first inlet port 112, a first outlet port 114, a second inlet port 116 and a second outlet port 118. The clamping valve assembly also includes a first tube 120 that fluidly connects the first inlet port 112 to the first outlet port 114, and a second tube 122 that fluidly connects the second inlet port 116 to the second outlet port 118. Other details of the clamping valve assembly are described below.

The housing 102 also may include a user interface 124 or an electronics port (e.g., USB or serial port) to connect to a remote user interface or to other system components (e.g., a compressed gas module, an anesthesia module, sensors, or the like). The control electronics 108 also may be provided remotely from the housing 102 and connected by a suitable electronics port.

The blower 106 has a blower inlet 126 and a blower outlet 128. The blower inlet 126 is fluidly connected to a blower inlet pipe 130, which preferably extends to a point where it is accessible from outside the housing 102. The blower outlet 128 is fluidly connected to the first inlet port 112 by a blower outlet pipe 132. One or more replaceable filters, such as HEPA (high efficiency particulate air) filters, and the like (not shown), may be included in the airflow paths to the blower inlet pipe 130 and/or the blower outlet 128, or elsewhere in the ventilator 100. The blower 106 preferably is provided in a sterile state, and the remainder of the ventilator 100 can also be provided in a sterile state.

The first outlet port 114, second inlet port 116 and second outlet port 118 are accessible from outside the housing 102. For example, the first outlet port 114 and second inlet port 116 may protrude from the housing 102, and the second outlet port 118 may be connected to an outlet port pipe 134 that extends outside the housing. Other alternatives and embodiments will be apparent to persons of ordinary skill in the art in view of the present disclosure.

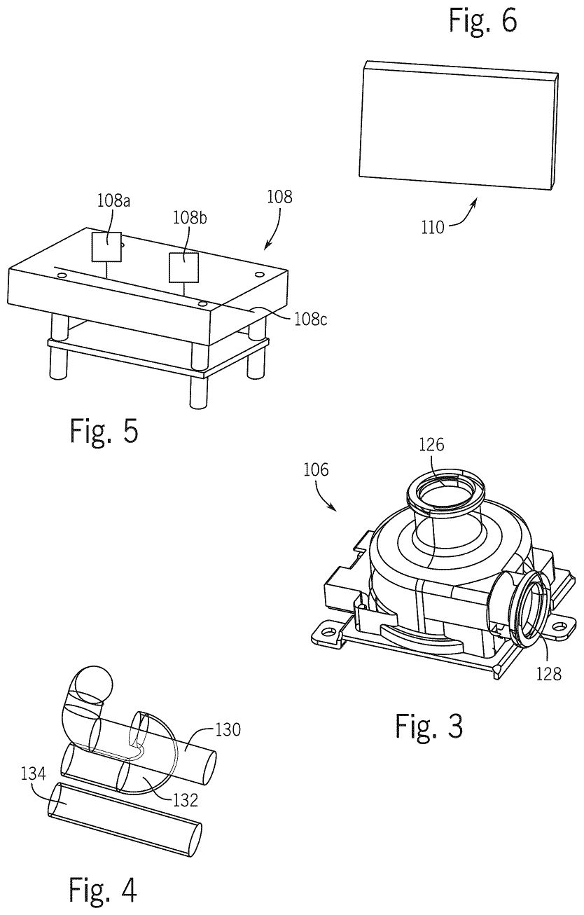

FIG. 3 shows the blower 106 in more detail. The blower 106 may comprise any suitable fan assembly for generating a flow of gas to a patient using the ventilator 100. For example, the blower 106 may be a double-port fan, a side channel blower, a multiple-stage radial blower, or the like. The blower has an associated motor, such as a brushless DC motor, that is configured to drive the blower's impeller at variable speeds to regulate the flow rate. The blower 106 preferably is secured to the housing 102 to be fully contained therein, but this is not strictly required. Such blowers are known in the art, and need not be described in more detail herein.

FIG. 4 shows the blower inlet pipe 130, blower outlet pipe 132, and outlet port pipe 134 removed from the ventilator housing 102. The blower inlet pipe 130, blower outlet pipe 132, and outlet port pipe 134 may comprise rigid or flexible plastic or elastomeric pipes, and may be opaque or transparent. One or more of the blower inlet pipe 130, blower outlet pipe 132, and outlet port pipe 134 may be joined together as a single unit. The selection of suitable pipe materials and dimensions will be understood by those skilled in the art, and need not be described in more detail herein.

FIG. 5 shows the control electronics 108 in more detail. The control electronics may include any suitable arrangement of electronic hardware, firmware, software and so on, to effectuate control of the blower 106 and clamping valve assembly 104. For example, the control electronics 108 may include a processor 108a, a memory 108b storing executable instructions, and a communication bus 108c for communicating between the processor 108a, memory 108b, power supply, clamping valve assembly 104, blower 106, and user interface 124. The control electronics also may include a programming port for receiving updates, a wireless interface, and so on. The control electronics 108 preferably are secured to the housing 102 to be fully contained therein, but some or all of the control electronics 108 may be remote from or external to the housing 102, and connected to the remainder of the ventilator 100 via suitable electrical connections. Such control electronics are generally conventional and need not be described in more detail herein.

The ventilator 100 may be operated by a local operator, remote monitoring, telemedicine, or a combination of techniques. For example, the ventilator control electronics 108 may include wireless connectivity for remote monitoring and control (telemedicine), in which case a remote expert could adjust ventilator control settings and alarms, and monitor airway parameters (e.g., respiratory rate, flow, pressure, volume) and patient vitals (e.g., heart rate, SpO2, ETCO2). A remote expert may also be able to communicate via audio or video with the in-person operator of the ventilator. There could also be one single remote operator that is controlling many ventilators at once, which would make the ventilator 100 ideal for austere environments and military applications or mass-casualty scenarios like COVID-19 or other pandemic or epidemics. Wireless connectivity could also be used for uploading remote firmware updates and new control algorithms, downloading operation data logs (both of the patient and the device's internal operations), and so on.

FIG. 6 shows the battery 110. The battery 110 may comprise a lithium-ion battery, or a battery using other chemistry to store an electric charge. The battery 110 may be replaced by or supplemented with one or more alternative energy supply devices, such as capacitors, a power cord to receive AC line voltage and a suitable transformer to control the power supply, and so on. Such batteries and other power supplies are known in the art, and need not be described in more detail herein.

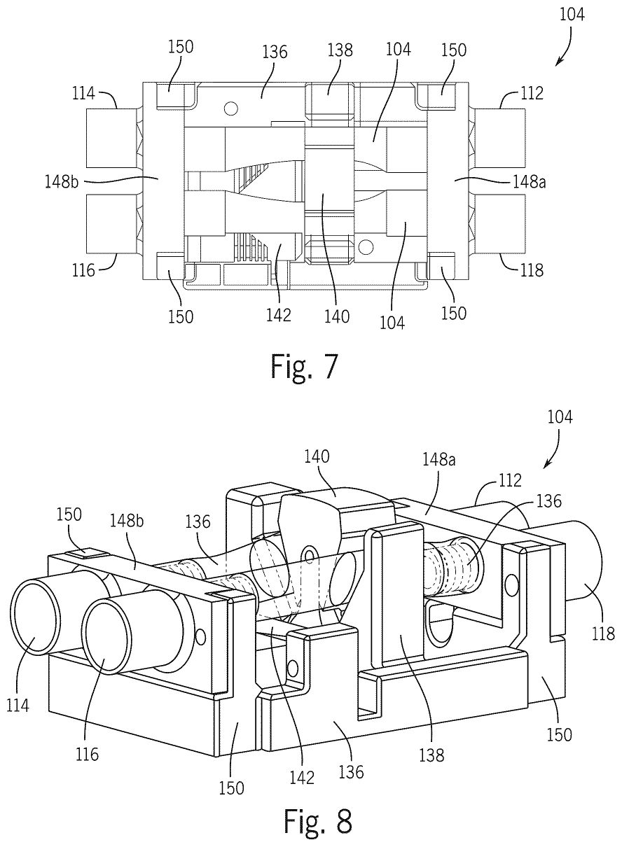

FIGS. 7 and 8 show the clamping valve assembly 104 removed from the housing 102. The clamping valve assembly 104 generally includes: a base 136; a clamp cradle 138; a clamp 140; a motor 142; a plurality of ports comprising: the first inlet port 112, first outlet port 114, second inlet port 116, and second outlet port 118; the first tube 120; and the second tube 122. The clamping valve assembly 104 preferably is removably mounted to the housing 102 by screws, clips, pins, or other fasteners. The clamping valve assembly 104 also may be removable from the housing 102 without removing any other components of the ventilator 100, but this is not strictly required.

The base 136 may comprise a rigid plastic or metal part formed by casting, injection molding, 3D printing, or other methods.

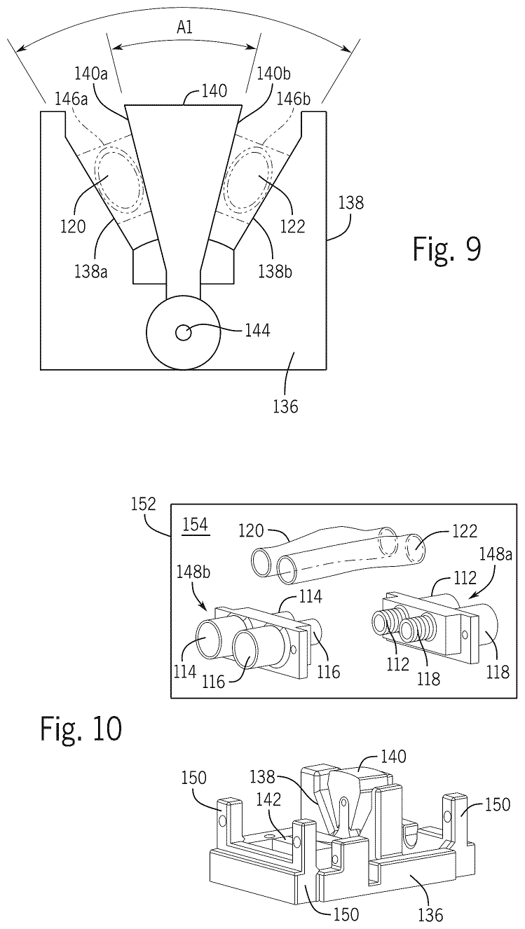

Further details of the clamping valve assembly 104 are now described with additional reference to FIGS. 9 through 13.

The clamp cradle 138 is positioned on the base 136, and has a first cradle surface 138a and a second cradle surface 138b. The clamp cradle 138 may be integrally formed with the base 136, or formed as a separate part that is joined to the base 136. Similarly, the clamp cradle 138 may comprise parts that are assembled together, or a single unitary structure. For example, the cradle surfaces 138a, 138b may be integral with a unitary clamp cradle 138 structure, or they may be separately formed and joined together (e.g., coatings or plates applied to surfaces of the remainder of the clamp cradle 138). The clamp cradle 138 preferably comprises a relatively rigid structure that will not appreciably deform during normal operation.

The clamp 140 is movably mounted relative to the base 136. In this case, the clamp 140 is movably mounted relative to the base 136 by being mounted on a motor shaft 144 of the motor 142, and the motor 142 is mounted to the base 136 or mounted to the housing 102 at a fixed location relative to the base 136. The clamp 140 has a first clamp surface 140a and a second clamp surface 140b. The first clamp surface 140a faces the first cradle surface 138a to define a first volume 146a between them.

The second clamp surface 140b faces the second cradle surface 138b to form a second volume 146b between them. The first volume 146a and second volume 146b are the regions in which the first tube 120 and second tube 122 are positioned, respectively, during normal operation of the device.

The first volume 146a and second volume 146b, and the respective clamp and cradle faces that form the first and second volumes, may have any suitable shape for the purpose of acting on the tube 120, 122 as described herein. In this case, the first and second clamp surfaces 140a, 140b, and the first and second cradle surfaces 138a, 138b are planar, and the first and second volumes 146a, 146b have generally prismatic shapes. The first clamp surface 140a and second clamp surface 140b converge towards the rotation axis, which is defined by the motor shaft 144, at a first angle A1. The first angle A1 may be any suitable angle, such as an angle in the range of 10° to 60°, or within the range of 20° to 50°. Similarly, the first cradle surface 138 a and the second cradle surface 138b converge towards the rotation axis at a second angle A2. The second angle A2 may be any suitable angle, such as an angle in the range of 40° to 90°, or within the range of 50° to 80°.

In some embodiments, the first angle A1 and second angle A2 may converge to a common point, and may converge to the rotation axis defined by the motor shaft 144. However, this is not strictly required. In other embodiments, the first angle A1 and second angle A2 may converge to different points, and the rotation axis may be above, between or below the point or points of convergence. For example, FIGS. 14 and 15 show a rotation axis located above a common point of convergence of angle A1 and A2. Other alternatives and embodiments will be apparent to persons of ordinary skill in the art in view of the present disclosure.

The clamp 140 and clamp surfaces 140a, 140b may comprises a unitary structure, or an assembly of parts. For example, the clamp surfaces 140a, 140b may be attached as plates or coatings to a central body forming the remainder of the clamp 140. The clamp 140 preferably comprises a relatively rigid structure that will not appreciably deform during normal operation. In the present case, the clamp 140 is provided as an assembly of a main clamp body 140c forming the first clamp surface 140a and the second clamp surface 140b, and a clamp drive arm 140d that is secured to the motor shaft 144 (e.g. by press fitting, welding, a fastener or key, or the like).

The clamp drive arm 140d preferably nests within the main clamp body 140c, and the two may be joined by a fastener such as a rivet or a screw. This arrangement may facilitate installation and removable of the first clamp surface 140a and second clamp surface 140b, without affecting the relatively rigid connection between the clamp drive arm 140d and the motor shaft 144. Other alternatives and embodiments will be apparent to persons of ordinary skill in the art in view of the present disclosure.

The motor 142 may be any motor suitable for providing the desired reciprocating motion described herein. For example, the motor 142 may comprise a servo motor which allows positive and accurate control of the motor position and rotation rate. The motor also may include limit switches or other features to prevent over-rotation or provide a record of the motor position. The motor 142 also may comprise an electromagnet or solenoid to provide motive force. Other alternatives and embodiments will be apparent to persons of ordinary skill in the art in view of the present disclosure.

The ports 112, 114, 116, 118 are shaped and dimensioned to provide a respiratory airflow, and may be formed to facilitate connection with other hoses, pipes, or the like. The ports 112, 114, 116, 118 may be formed of rigid plastic or metal, and may be opaque or transparent. The ports 112, 114, 116, 118 may be integrally formed with the base 136 or otherwise permanently attached to the base 136, but more preferably one or more of the ports 112, 114, 116, 118 is selectively removable from the base 136. For example, in the shown embedment, the first inlet port 112 and second outlet port 118 are joined together as a first port block 148a that is removably mounted to the base 136, and the first outlet port 114 and second inlet port 116 are joined together as a second port block 148b that is removably mounted to the base 136. The first port block 148a and second port block 148b may be formed by integrally molding the respective ports 112, 114, 116, 118 together with a connecting structure, or by attaching the respective ports 112, 114, 116, 118 or portions of the ports 112, 114, 116, 118 to an intermediate connecting structure. In other cases, the ports 112, 114, 116, 118 may be connected together in different arrangements. For example, all four ports 112, 114, 116, 118 may be connected together as a single port block. As another example, the first inlet port 112 and first outlet port 114 may be connected together as a first port block, and the second inlet port 116 and second outlet port 118 may be connected together as a second port block. Other alternatives and embodiments will be apparent to persons of ordinary skill in the art in view of the present disclosure.

The first and second port blocks 148a, 148b may be secured to the base 136 by any suitable connection. For example, each port block 148a, 148b may be connected between a respective pair of posts 150 extending upwards from the base 136. Such connection may be by mechanical fasteners (e.g., screws or rivets), snap fitting elements, and so on. The first and second port blocks 148a, 148b preferably can be removed from the remainder of the clamping valve assembly 104 and/or the ventilator housing 102 without removing any other parts other than the first and second tubes 120, 122, but this is not strictly required.

As best shown in FIG. 9, a portion of the first tube 120 is located in the first volume 146a, and a portion of the second tube 122 is located in the second volume 146b. The first tube 120 and second tube 122 are selectively compressed to facilitate respiratory breathing, and to this end, the first tube 120 and the second tube 122 each is flexible, at least in the respective region within the first volume 146a and the second volume 146b. Other portions of the first tube 120 and the second tube 122 may be rigid. For example, each tube 120, 122 may terminate at rigid hose fittings. However, for cost and convenience, it may be preferable in some cases to make the first tube 120 and second tube 122 as continuous, uniform flexible tubes. The flexible portions of the first tube 120 and second tube 122 may have any dimensions suitable to use as a ventilator. For example, the first tube 120 and second tube 122 may comprise soft latex rubber tubes having a diameter of 0.5 inches and a wall thickness of ⅙ inch.

Other tube dimensions will be readily appreciated by persons of ordinary skill in the art in view of the present disclosure.

The first tube 120 and second tube 122 may be separately removable from the remainder of the clamping valve assembly 104 and/or the ventilator housing 102, preferably without otherwise disassembling parts of the ventilator 100. The first tube 120 and second tube 122 also may be removable with the respective ports 112, 114, 116, 118 to which they are connected. For example, the first tube 120 and second tube 122 may be secured to the first port block 148a and second port block 148b, and these parts may be removable as a single unit from the remainder of the clamping valve assembly 104 and the ventilator 100.

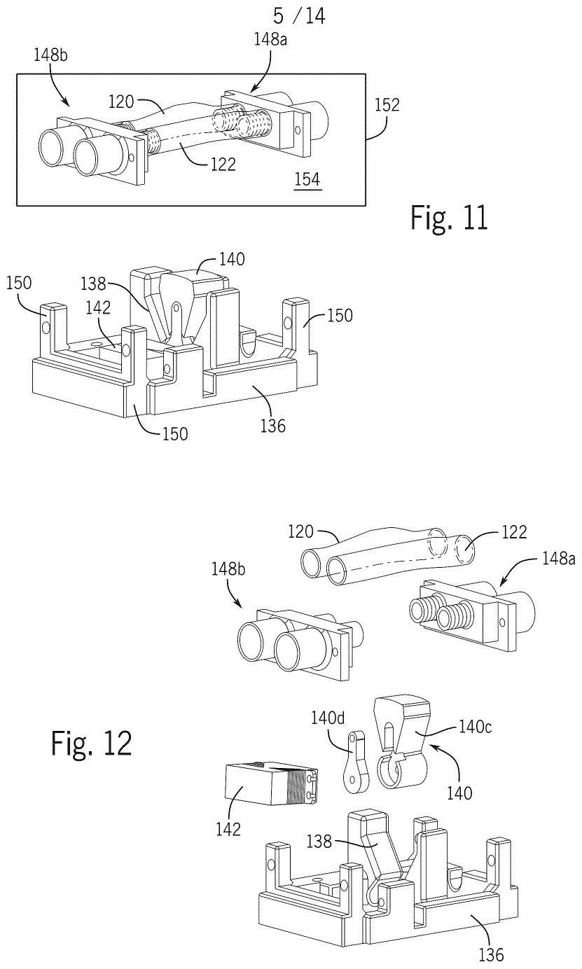

The present arrangement is expected to provide a significant advantage with respect to servicing the ventilator 100. For example, the ports 112, 114, 116, 118 and tubes 120, 122 may be provided in a sterile state within a sealed package 152 comprising a sterile interior volume 154, such as shown in FIGS. 10 and 11. Other replaceable components, such as a replacement blower 106, blower inlet pipe 130, blower outlet pipe 132 outlet port pipe, and air filters, could also be provided in the same sealed package 152. For example, the blower inlet pipe 130 and blower outlet pipe 132 may be provided in a sterile sealed package 152 with an air filter pre-assembled to each. Also, multiple sealed packages also may be used to hold the replaceable parts. The ports 112, 114, 116, 118 may be assembled with the tubes 120, 122 as shown in FIG. 11, or separate, as shown in FIG. 10. Other alternatives and embodiments will be apparent to persons of ordinary skill in the art in view of the present disclosure.

The ventilator 100 can be serviced by opening the housing 102, removing the used ports 112, 114, 116, 118 and tubes 120, 122, opening the sealed package 152, and installing the new sterile ports 112, 114, 116, 118 and tubes 120, 122. This process is further facilitated by using port blocks 148a, 148b, and by preassembling the parts as shown in FIG. 11. This service preferably can be done without otherwise removing parts of the ventilator 100. For example, the remainder of the clamping valve assembly 104 and other parts described preferably herein can remain in place while the ports 112, 114, 116, 118 and tubes 120, 122 are removed and replaced.

Such servicing is further facilitated by forming the blower outlet pipe 132 and outlet port pipe 134 from flexible materials to allow them to be easily removed and installed from the first inlet port 112 and the second outlet port 118, respectively. Also, such servicing can be facilitated by forming the first outlet port 114 and second inlet port 116 to extend from the housing 102 through one or more openings or be located near the housing wall, so that they can be directly connected to external hoses of the ventilator assembly. Such ease in servicing is expected to improve the utility of the ventilator, reduce operating costs, and allow for rapid servicing to transfer the ventilator between patients.

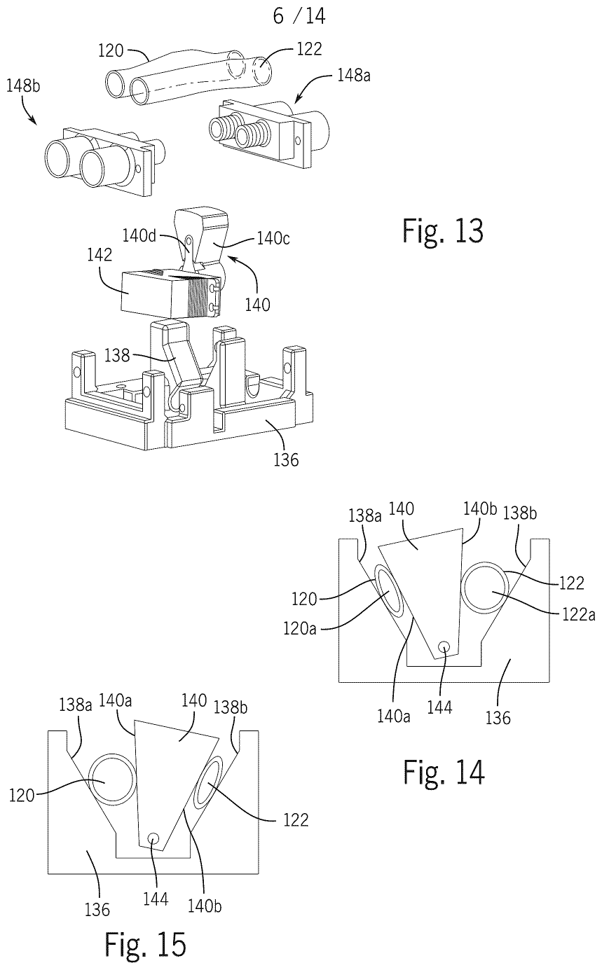

The clamping valve assembly 104 also may be constructed to allow relatively simple and cost-effective manufacture and assembly. For example, FIG. 12 shows that the clamping valve assembly 104 can have a small number of working parts, and FIGS. 10-13 11 show how the parts can be assembled together in a few steps, such as:
Step 1: assemble the motor 142 and clamp 140 together as shown from FIG. 12 to FIG. 13;
Step 2: install the motor 142 and clamp 140 assembly to the base 136 as shown from FIG. 13 to FIG. 10;
Step 3: assemble the tubes 120, 122 to port blocks 148a, 148b as shown from FIG. 10 to FIG. 11; and
Step 4: install the tube and port block assembly to the remainder of the clamping valve assembly by fitting the tubes 120, 122 to their respective spaces between the clamp 140 and the clamp cradle 138, and attaching the port blocks 148a, 148b to the respective posts 150 as shown in FIG. 8.

Referring now to FIGS. 14 and 15, the operation of the clamping valve assembly 104 is shown in more detail. As noted above, the motor 142 is operative to rotate the clamp 140 relative to the base 136. When operated in one direction (clockwise as seen in the Figures), the motor 142 moves the second clamp surface 140b towards the second cradle surface 138b, to thereby move the clamp 140 from a first clamp position as shown in FIG. 14, to a second clamp position as shown in FIG. 15. When operated in the other direction (counterclockwise as seen in the Figures), the motor 142 moves the first clamp surface 140a towards the first cradle surface 138b, to move the clamp 140 from the second clamp position back to the first clamp position. This process is repeated according to the patient's respiration rate.

The patient's respiration rate itself can be defined in many ways. For example, the ventilator 100 may implement a mandatory respiration rate that triggers for example 20 breaths per minute of constant volume/pressure. Alternatively, the ventilator 100 can implement a patient-triggered respiration rate, such as by using a flow sensor to detect when the patient attempts to initiate a breath (either because of increased flow or a dip in pressure) and correspondingly start the breath. A combination of the above can also be used to establish the patient's respiration rate.

The ventilator 100 also could operate in a CPAP mode where a continuous constant pressure is delivered to the patient while the patient breathes on their own and the clamping valve assembly remain at a fixed position. The ventilator 100 preferably can be operated in these or a variety of alternative modes.

In the first clamp position, the first volume 146a is smaller (i.e., smaller in volumetric size) than the second volume 146b. In this position, the first tube 120 is compressed to a relatively large degree (as compared to the second tube 122) to reduce the size of the first tube's internal passage 120a (and possibly close it completely). Meanwhile, the second tube 122 is either not compressed or, more preferably, is compressed by a relatively small amount (as compared to the first tube 120), so that the second tube's internal passage 122a is relatively open (as compared to the first tube's internal passage 120a) to allow a relatively large volumetric flow rate therethrough.

In the second clamp position, the second volume 146b is smaller than the first volume 146a. In this position, the second tube 122 is compressed to a relatively large degree (as compared to the first tube 120) to reduce the size of the second tube's internal passage 122a (and possibly close it completely). Meanwhile, the first tube 120 is either not compressed or, more preferably, is compressed by a relatively small amount (as compared to the second tube 122), so that the first tube's internal passage 120a is relatively open (as compared to the second tube's internal passage 122a) to allow a relatively large volumetric flow rate therethrough.

In use, the motor 142 is operated to reciprocate between the first clamp position and the second clamp position, and the blower 106 is operated continuously or cyclically to generate a positive-pressure flow of gas through the first tube 120 and to the patient. The motor 142 may operate to move the clamp 140 according to any desirable movement pattern. For example, the motor 142 may move the clamp 140 at an essentially constant speed as it moves between the first and second clamp position, and stop at the first and second clamp positions for a predetermined or variable time before moving back to the other position. Other alternatives and embodiments will be apparent to persons of ordinary skill in the art in view of the present disclosure.

Preferably, the particular selection of the motion pattern can be adjusted as desired for the particular application using inputs to the user interface 124. The selection of a suitable movement pattern will be within the skill of the person of ordinary skill in the art in view of the present disclosure, and need not be described in detail herein.

It is expected that embodiments of the clamping valve assembly 104 may provide various improvements to ventilator systems. For example, the clamping valve assembly 104 can be made as a modular unit, can be easily serviced and can be manufactured at relatively little cost. The clamping valve assembly 104 can also be made relatively small, leading to a smaller and lighter ventilator 100.

Another distinct advantage is that the clamping valve assembly 104 can be constructed such that the clamp 140 at least partially compresses both the first tube 120 and the second tube 122 at all positions of the clamp 140. This construction allows the clamp 108 to operate using a relatively short stroke, and in operation it will always be both increasing and decreasing flow through the first tube 120 and second tube 122 at the same time.

Embodiments are also expected to create a more linear relationship between the position of the valve, the fan speed (as controlled by power input), and the vent pressure in the circuit, which allows more rapid and more accurate pressure changes. Thus, the ventilator 100 can be configured to easily deliver volume control-shaped waveforms to the patient by linearly increasing the position of the valve throughout the inhale duration of the breathing cycle. For example, the clamping valve assembly 104 can be operated to provide a linear increase in pressure and volume flow rate and a relatively constant flow during the inhale duration of the breathing cycle. The clamping valve assembly can also be operated to provide a square waveform for the volume flow rate and pressure, with a quick spike in the flow at the beginning of the inhale cycle.

Other controlled movements are also available, and other options will be apparent to persons of ordinary skill in the art in view of the present disclosure.

A ventilator 100 incorporating the clamping valve assembly 104 can also be configured to provide a feed-back based control system, in which the ventilator 100 can be fully controlled using peripheral oxygen saturation and end-tidal CO₂ measurements to automatically adjust the ventilator settings.

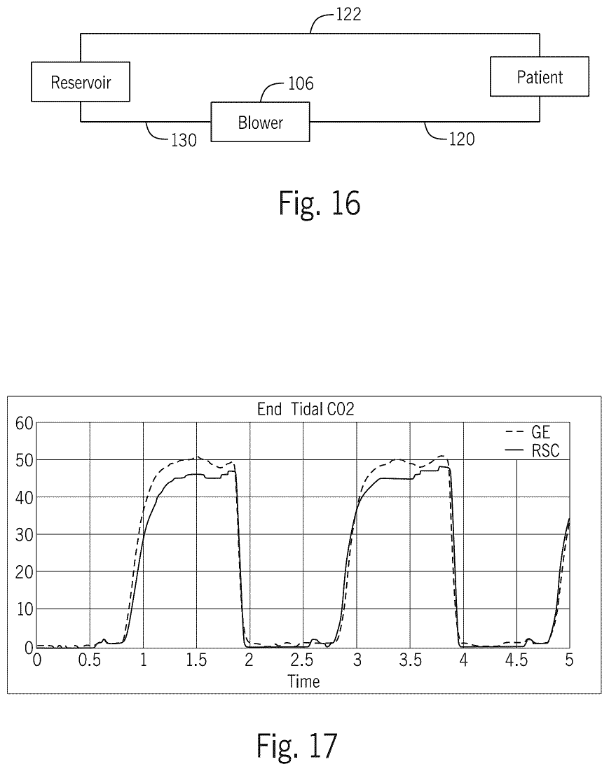

Referring to FIG. 16, yet another advantage of a ventilator 100 incorporating the clamping valve assembly 104 is that there is a single point of entry at the blower 106 (i.e., blower inlet pipe 130). Still further, the blower 106 is not able to generate a negative pressure on the patient's lungs if there is a software or hardware malfunction.

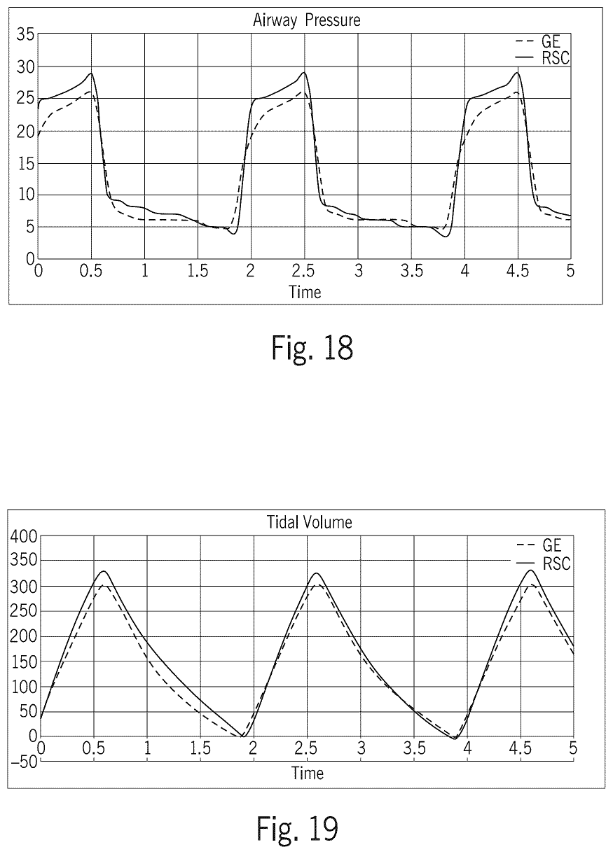

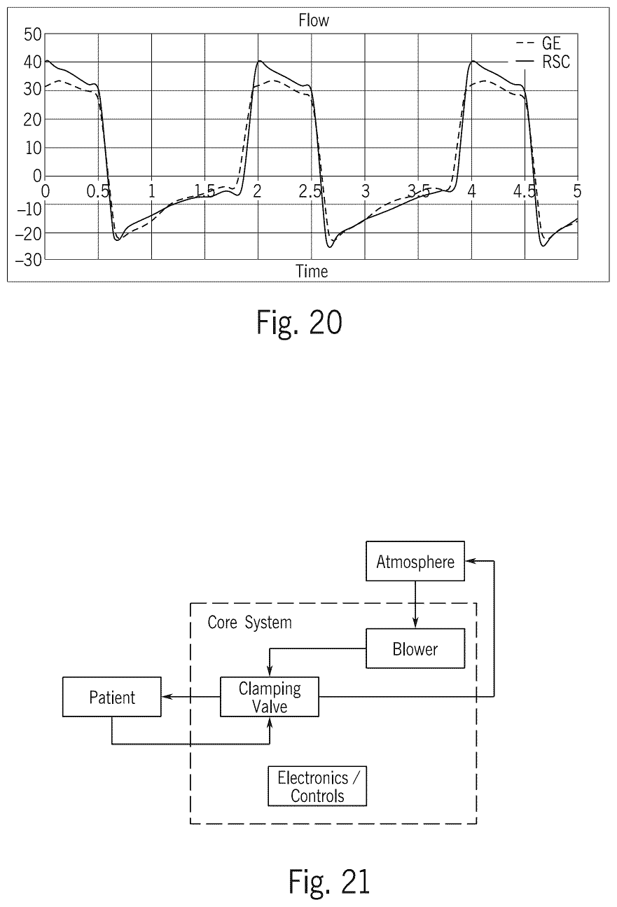

It has been found that embodiments of a ventilator 100 as described herein can provide suitable performance, while also being contained in a relatively lightweight and serviceable package. Examples of operating results are shown in FIGS. 17 through 20. These plots illustrate the performance of an exemplary embodiment (solid line) as compared to a commercially-available ventilator (broken line). FIG. 17 illustrates end-tidal CO₂, FIG. 18 illustrates airway pressure, FIG. 19 illustrates tidal volume, and FIG. 20 illustrates flow rate.

Referring back to FIG. 1, one or more of the measured operating variables can be displayed, in real time or upon being recalled from memory, on the user interface 124. For example, the user interface 124 can be configured to display one or more of: a pressure measurement of a gas passing through the first tube 120 and/or the second tube 122; a peak flow rate measurement of a gas passing through the first tube 120 and/or the second tube 122; an end tidal carbon dioxide measurement; and a peak inspiratory pressure measurement. These measurements may be taken using any suitable sensor positioned at any suitable location. For example, end tidal carbon dioxide and/or peak inspiratory pressure measurements can be taken proximal to the patient using a proximal flow and pressure sensor. As another example, a bidirectional proximal flow sensor may be used for flow and tidal volume measurements. Also, a separate pressure sensor line may be connected from a location proximal to the patient to a sensor that is inside the ventilator body. The user interface 124 also may be configured to illustrate other operating conditions, such as tidal volume (e.g., by integrating net flow found by subtracting inspired flow minus expired flow, or with a bidirectional proximal flow sensor), fault conditions, battery power status, and so on.

A ventilator 100 such as described herein can be used in a variety of different ventilator system configurations. Examples of various alternatives are shown in FIGS. 21 through 24.

FIG. 21 shows a ventilator system in its most basic form, with the blower receiving atmospheric air.

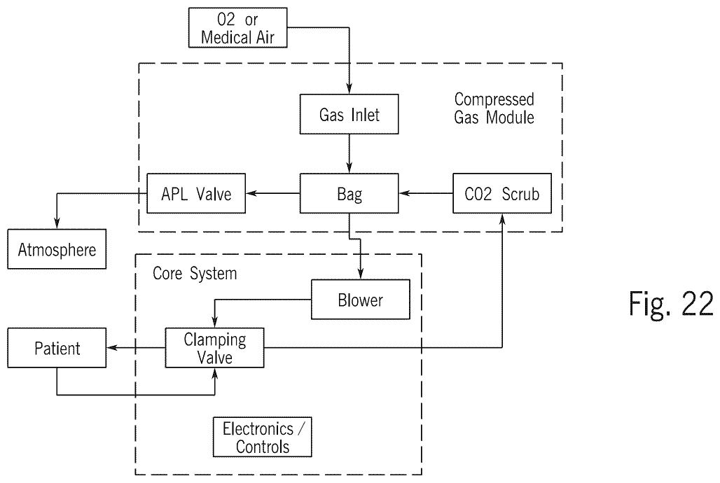

FIG. 22 shows a ventilator system having an added compressed gas module comprising, for example: a carbon dioxide scrubber having a scrubber inlet in fluid communication with the second outlet port, and a scrubber outlet; a ventilator bag having a first bag inlet in fluid communication with the scrubber outlet, a bag outlet in fluid communication with the blower inlet, and a second bag inlet; a gas inlet port configured to connect a supply of oxygen and/or a supply of medical air to the ventilator bag; and an adjustable pressure-limiting valve configured to vent gas from the ventilator bag to an atmosphere. The gas inlet may include a pressure regulator and/or flow regulator to ensure that the gas being delivered to the system is at a controlled flow rate and safe pressure. This flow rate could be controllable by the user or set automatically by the device control system. The adjustable pressure-limiting valve could be set to a constant limiting pressure or it could be adjustable by the user or automatically adjusted by the device control system. It will be understood that the APL valve should never exceed the patient's positive end expiratory pressure (PEEP) to ensure that the reservoir pressure is lower than the patient lung pressure and gas will flow out of the lungs and not backward.

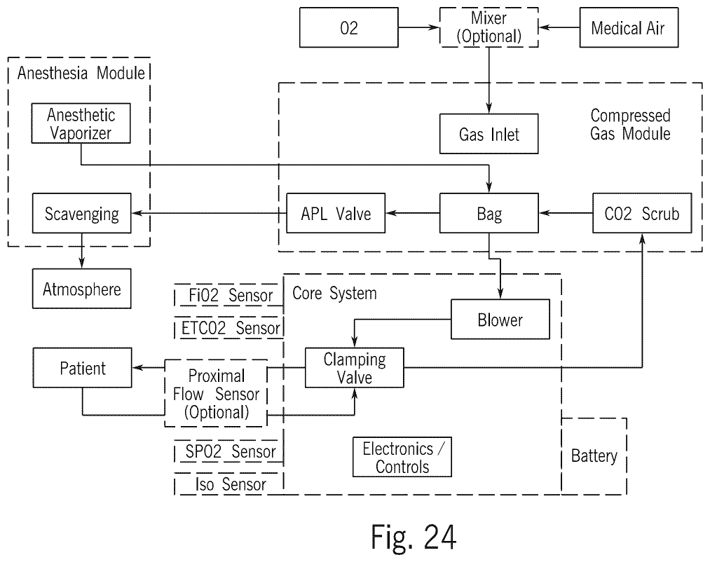

The compressed gas module (or portions of it) can be integrated into the core ventilator 100. Alternatively, the entire compressed gas module may be a separate modular unit that is removable from the ventilator 100, such as by selectively disconnecting the scrubber inlet from the second outlet port 118 and the ventilator bag from the blower inlet 126, and disconnecting any associated electronic connections. The compressed gas module can deliver compressed gases such as oxygen or medical air, or a combination of both by using a mixer (see, e.g. FIG. 24). The carbon dioxide scrubber removes excess carbon dioxide from the gaseous mixture, which provides a closed-loop setup to reduce compressed gas waste.

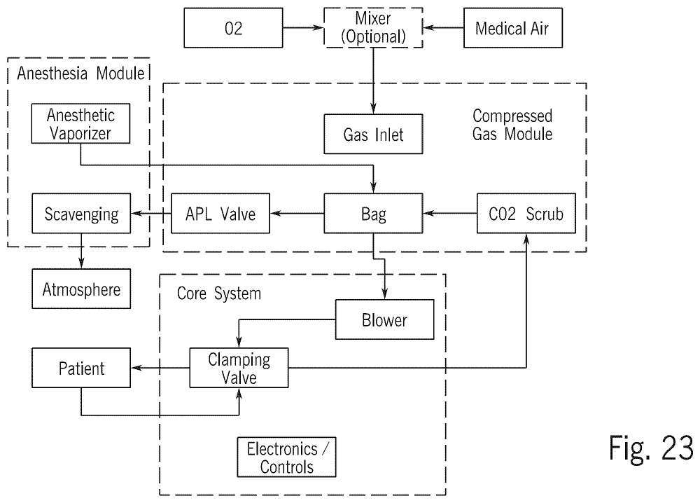

A ventilator system also may be provided with a compressed gas module and an anesthesia module, such as shown in FIG. 23. Here, the anesthesia module includes an anesthetic vaporizer in fluid communication between the gas inlet port and the ventilator bag, and a scavenging system in fluid communication between the adjustable pressure-limiting valve and the atmosphere. The anesthetic vaporizer may be partially or fully incorporated into the ventilator 100 or compressed gas module, or provided as a separate removable unit. For example, the anesthetic vaporizer may be selectively disconnectable from the gas inlet port and the ventilator bag, the scavenging system may be selectively disconnectable from the adjustable pressure-limiting valve and the atmosphere, and any associated electronic connections between the ventilator 100 and the anesthesia module may be selectively disconnectable.

Referring to FIG. 24, a ventilator system also may include various sensors 31. Examples of sensors include, but are not limited to: a fraction of inspired oxygen (FiO2) sensor; an end tidal carbon dioxide sensor; a proximal flow sensor; a peripheral oxygen saturation (SpO2) sensor; and an isoflurane sensor. One or more of these sensors may be integrated into the ventilator 100, or provided separately and connected by electrical wires or the like. As with other operating parameters, measurements from such sensors could be displayed on the display of the user interface 124.

These or other embodiments also may be configured to interact with other sensors and systems. For example, the ventilator 100 may be configured to interface with a brain monitoring device as an additional module. Such a device could be used to collect patient vitals, including intracranial pressure (ICP), cerebral blood flow (CBF), and cerebral oxygenation (StO2). These additional metrics could be used as feedback to an automatic control algorithm operated by the control electronics 108 or to trigger alarms that alert the physician and help guide the physician with adjusting the ventilator's control settings. For example, elevated ICP is a major consequence of severe traumatic brain injury (TBI), causing increased risk of brain herniation and worsened outcomes. Elevated ICP can be reduced through controlled intervals of hyperventilation. However, prolonged hyperventilation can lead to dangerously low CBF, furthering cerebral ischemia/acute brain injury. By measuring CBF and StO2, hyperventilation treatment can be modulated, maintaining a safe oxygenation range and possibly mitigating the risk of cerebral ischemia/acute brain injury. These and other algorithms and control systems may be integrated into the ventilator control electronics 108, or the ventilator control electronics 108 may be configured to receive controls from a separate system implementing such algorithms and control systems.

Other alternatives and embodiments will be apparent to persons of ordinary skill in the art in view of the present disclosure.

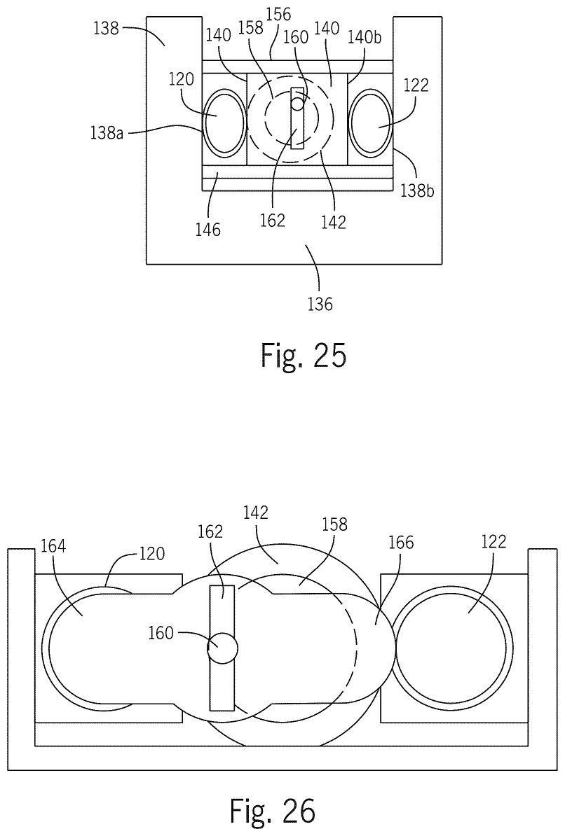

Referring to FIG. 25, another embodiment of a clamping valve assembly 104 is shown. In this case, the clamping valve assembly is essentially the same as the embodiment of FIG. 8, but the rotating clamp 140 is replaced by a clamp 140 that slides along a linear path, as constrained by tracks 156 or the like. Here, the motor 142 drives a drive plate 158 or arm having an eccentric pin 160. As the motor 142 rotates the drive plate 158, the eccentric pin 160 moves in a circular path. The eccentric pin 160 is located in a drive slot 162 in the clamp 140. Thus, motor 142 rotation caused the clamp 140 to move back and forth along the tracks 156. Other embodiments may use other drive arrangements to operate the clamp 140, and other alternatives and embodiments will be apparent to persons of ordinary skill in the art in view of the present disclosure.

FIG. 26 illustrates another embodiment of a clamping valve assembly 104. In this case, the base 136, has a first tube 120 and a second tube 122, which are both rigid. The first tube 120 has a first valve body 164 that is configured to move to thereby change the cross-sectional area of the first tube 120, and the second tube 122 has a second valve body 166 that is configured to move to thereby change the cross-sectional area of the second tube 122. The first valve body 164 and second valve body 166 may comprise any suitable valve structure. In this case, the first valve body 164 and second valve body 166 comprise two ends of a single integral sliding plate that intersects the first tube 120 and second tube 122, with sliding seals to provide an air-tight fit between the valve bodies 164, 166 and tubes 120, 122. Control electronics 108 drive the valve bodies 164, 166 via a motor 142 and driving arrangement, such as the eccentric drive 158, 160, 162 described above, a linear actuator (e.g., solenoid or electromagnetically operated piston), or the like.

In use, the control electronics 108 are configured to operate the one or more motors to simultaneously move the first valve body to reduce the cross-sectional area of the first tube and move the second valve body to increase the cross-sectional area of the second tube; and operate the one or more motors to simultaneously move the first valve body to increase the cross-sectional area of the first tube and move the second valve body to decrease the cross-sectional area of the second tube. This operation is performed repeatedly according the patient's respiration rate.

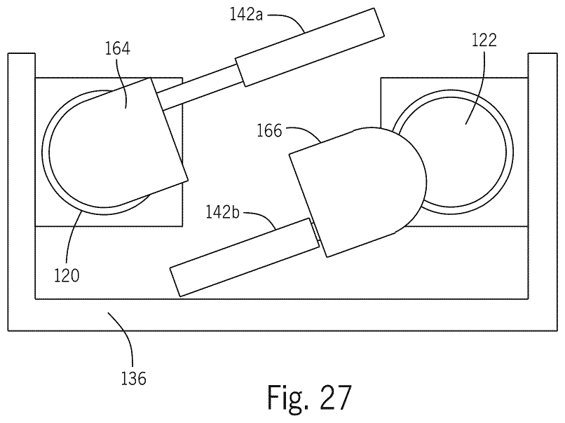

FIG. 27 illustrates another embodiment, similar to the one of FIG. 26. In this case, however, the first valve body 164 and second valve body 166 are operated separately by respective first and second motors 142a, 142b (in this case, linear actuators). With this arrangement, the control electronics 108 can operate the first motor 142a and second motor 142b in synchronization to move the first valve body 164 and second valve body 166 as described above in relation to FIG. 26. In addition, the control electronics 108 can operate the first motor 142a and the second motor 142b separately, to achieve more complicated movements of the first valve body 164 and the second valve body 166. For example, the control electronics 108 can operate the first motor 142a and the second motor 142b to simultaneously not move the first valve body 164 to maintain the cross-sectional area of the first tube 120 at a constant value and move the second valve body 166 to change the cross-sectional area of the second tube 122; and/or operate the first motor 142a and the second motor 142b to simultaneously move the first valve body 164 to change the cross-sectional area of the first tube 120 and not move the second valve body 166 to maintain the cross-sectional area of the second tube 122 at a constant value. Other alternatives and embodiments will be apparent to persons of ordinary skill in the art in view of the present disclosure.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” “includes,” “including,” or any other variation thereof, are intended to cover a non-exclusive inclusion, such that a process, method, article, or apparatus that comprises or includes a list of elements or steps does not include only those elements or steps but may include other elements or steps not expressly listed or inherent to such process, method, article, or apparatus. An element preceded by “a” or “an” does not, without further constraints, preclude the existence of additional identical elements in the process, method, article, or apparatus that comprises the element.

Unless otherwise stated, any and all measurements, values, ratings, positions, magnitudes, sizes, and other specifications that are set forth in this specification, including in the claims that follow, are approximate, not exact. Such amounts are intended to have a reasonable range that is consistent with the functions to which they relate and with what is customary in the art to which they pertain. For example, unless expressly stated otherwise, a parameter value or the like may vary by ±10% from the stated amount.

In addition, in the foregoing Detailed Description, it can be seen that various features are grouped together in various examples for the purpose of streamlining the disclosure. This method of disclosure is not to be interpreted as reflecting an intention that the claimed examples require more features than are expressly recited in each claim. Rather, as the following claims reflect, the subject matter to be protected lies in less than all features of any single disclosed example. Thus, the following claims are hereby incorporated into the Detailed Description, with each claim standing on its own as a separately claimed subject matter.

While the foregoing has described specific examples, it is understood that various modifications may be made therein and that the subject matter disclosed herein may be implemented in various forms and examples, and that they may be applied in numerous applications, only some of which have been described herein. It is intended by the following claims to claim any and all modifications and variations that fall within the true scope of the present concepts.

1. A ventilator clamping valve assembly comprising:
a base;
a clamp cradle positioned on the base and having a first cradle surface and a second cradle surface;
a clamp movably mounted relative to the base, the clamp having a first clamp surface facing the first cradle surface to define a first volume between the first clamp surface and the first cradle surface, and a second clamp surface facing the second cradle surface to define a second volume between the second clamp surface and the second cradle surface;
a motor operatively connected to the clamp and configured to move the clamp between a first clamp position in which the first volume has a starting first volume value and the second volume has a starting second volume value, and a second clamp position in which the first volume has a final first volume value that is different from the starting first volume value, and the second volume has a final second volume value that is different from the starting second volume value;
a plurality of ports comprising: a first inlet port, a first outlet port, a second inlet port, and a second outlet port;
a first tube extending through the first volume and fluidly connecting the first inlet port and the first outlet port; and
a second tube extending through the second volume and fluidly connecting the second inlet port and the second outlet port.

2. The ventilator of claim 1, wherein the clamp cradle is integrally formed with the base.

3. The ventilator of claim 1, wherein the clamp is mounted to the base to rotate about a rotation axis.

4. The ventilator of claim 1, wherein the clamp is mounted to the base to slide along a linear path.

5. The ventilator of claim 4, wherein the clamp is mounted to the base by an output shaft of the motor, and the output shaft defines the rotation axis.

6. The ventilator of claim 4, wherein the first clamp surface and the second clamp surface converge towards the rotation axis.

7. The ventilator of claim 5, wherein the first cradle surface and the second cradle surface converge towards the rotation axis.

8. The ventilator of claim 7, wherein the first clamp surface and the second clamp surface converge at a first angle of 10° to 60°, and the first cradle surface and the second cradle surface converge at a second angle of 40° to 90°.

9. The ventilator of claim 7, wherein the first clamp fact and the second clamp surface converge at a first angle of 20° to 50°, and the first cradle surface and the second cradle surface converge at a second angle of 50° to 80°.

10. The ventilator of claim 1, wherein the clamp comprises an arm operatively connected to the motor, and a clamp body secured to the arm, the clamp body defining the first clamp surface and the second clamp surface.

11. The ventilator of claim 1, wherein the motor comprises a servo motor.

12. The ventilator of claim 1, wherein one or more of the plurality of ports is removably mounted to the base.

13. The ventilator of claim 1, wherein the first inlet port and the second outlet port are attached together as a first port block that is removably mounted to the base, and the first outlet port and the second inlet port are attached together as a second port block that is removably mounted to the base.

14. The ventilator of claim 13, wherein the first inlet port and the second outlet port are integrally formed with the first port block, and the first outlet port and the second inlet port are integrally formed with the second port block.

15. The ventilator of claim 1, wherein the first tube is selectively removable from the first inlet port and the first outlet port and/or the second tube is selectively removable from the second inlet port and the second outlet port.

16. The ventilator of claim 1, wherein the first tube and/or the second tube at least partially comprises a flexible tube having an inner diameter of 0.5 inches and a wall thickness of ⅙ inch.

17. The ventilator of claim 1, wherein:
when the clamp is in the first clamp position, the first tube is at least partially compressed between the first clamp surface and the first cradle surface and the second tube is at least partially compressed between the second clamp surface and the second cradle surface; and/or
when the clamp is in the second clamp position, the first tube is at least partially compressed between the first clamp surface and the first cradle surface and the second tube is at least partially compressed between the second clamp surface and the second cradle surface.

18. The ventilator clamping valve of claim 1, wherein the starting first volume value is greater than the starting second volume value, and the final first volume value is less than the final second volume value.

19. A ventilator system comprising:
a housing;
a clamping valve assembly connected to the housing and comprising:
a base,
a clamp cradle positioned on the base and having a first cradle surface and a second cradle surface,
a clamp movably mounted relative to the base, the clamp having a first clamp surface facing the first cradle surface to define a first volume between the first clamp surface and the first cradle surface, and a second clamp surface facing the second cradle surface to define a second volume between the second clamp surface and the second cradle surface, and
a motor operatively connected to the clamp and configured to move the clamp between a first clamp position in which the first volume has a starting first volume value and the second volume has a starting second volume value, and a second clamp position in which the first volume has a final first volume value that is different from the starting first volume value, and the second volume has a final second volume value that is different from the starting second volume value,
a plurality of ports comprising: a first inlet port, a first outlet port, a second inlet port, and a second outlet port,
a first tube extending through the first volume and fluidly connecting the first inlet port and the first outlet port, and
a second tube extending through the second volume and fluidly connecting the second inlet port and the second outlet port;
a blower having a blower inlet and a blower outlet, the blower outlet being fluidly connected to the first inlet port when the clamping valve assembly is connected to the housing;
a controller configured to operate the blower to generate a flow of gas into the first inlet, and to operate the motor to reciprocate the clamp between the first position and the second position.

20. The ventilator system of claim 19, further comprising a battery located in or connectable to the housing and configured to provide power to the controller.

21. The ventilator system of claim 19, further comprising a user interface configured to be operated by the controller to display one or more of:
a pressure measurement of a gas passing through the first tube and/or the second tube;
a peak flow rate measurement of a gas passing through the first tube and/or the second tube;
an end tidal carbon dioxide measurement;
a peak inspiratory pressure measurement;
a tidal volume measurement
a fraction of inspired oxygen (FiO2) measurement; and
an oxygen saturation (SpO2) measurement.

22. The ventilator system of claim 19, wherein the clamping valve assembly is removably connected to the housing.

23. The ventilator system of claim 19, wherein the first outlet and the second inlet extend through one or more openings through the housing to be accessible outside the housing, and a remainder of the clamping valve assembly is located within the housing.

24. The ventilator system of claim 19, wherein one or more of the plurality of ports is replaceable while a remainder of the clamping valve assembly is connected to the housing.

25. The ventilator system of claim 24, wherein the first inlet port and the second outlet port are attached to a first port block that is replaceable while a remainder of the clamping valve assembly is connected to the housing and/or the first outlet port and the second inlet port are attached to a second port block that is replaceable while a remainder of the clamping valve assembly is connected to the housing.

26. The ventilator system of claim 19, wherein the first tube is replaceable while a remainder of the clamping valve assembly is connected to the housing and/or the second tube is replaceable while a remainder of the clamping valve assembly is connected to the housing.

27. The ventilator system of claim 19, wherein the first tube and/or the second tube at least partially comprises a flexible tube having an inner diameter of 0.5 inches and a wall thickness of ⅙ inch.

28. The ventilator system of claim 19, further comprising a compressed gas module comprising:
a carbon dioxide scrubber having a scrubber inlet in fluid communication with the second outlet port, and a scrubber outlet;
a ventilator bag having a first bag inlet in fluid communication with the scrubber outlet, a bag outlet in fluid communication with the blower inlet, and a second bag inlet;
a gas inlet port configured to connect a supply of oxygen and/or a supply of medical air to the ventilator bag; and
an adjustable pressure-limiting valve configured to vent gas from the ventilator bag to an atmosphere.

29. The ventilator system of claim 28, wherein the compressed gas module further comprises a pressure regulator and/or a flow regulator.

30. The ventilator system of claim 28, wherein the scrubber inlet is selectively disconnectable from the second outlet port and the ventilator bag is selectively disconnectable from the first inlet port.

31. The ventilator system of claim of claim 28, further comprising a gas mixer fluidly connecting a supply of oxygen and a supply of medical air to the gas inlet port, the gas mixer configured to mix the oxygen and the medical air.

32. The ventilator system of claim 28, further comprising an anesthesia module comprising:
an anesthetic vaporizer in fluid communication between the gas inlet port and the ventilator bag; and
a scavenging system in fluid communication between the adjustable pressure-limiting valve and the atmosphere.

33. The ventilator system of claim 32, wherein:
the anesthetic vaporizer is selectively disconnectable from the gas inlet port and the ventilator bag; and
the scavenging system is selectively disconnectable from the adjustable pressure-limiting valve and the atmosphere.

34. The ventilator system of claim 19, further comprising one or more of:
a fraction of inspired oxygen (FiO2) sensor;
an end tidal carbon dioxide sensor;
a proximal flow sensor;
a peripheral oxygen saturation (SpO2) sensor; and
an isoflurane sensor.

35. The ventilator system of claim 19, wherein the starting first volume value is greater than the starting second volume value, and the final first volume value is less than the final second volume value.

36. A method for assembling or servicing a ventilator, the method comprising:
accessing an interior of a ventilator housing;
removing and replacing at least one of a first inlet port, a first outlet port, a second inlet port, and a second outlet port of a clamping valve assembly attached within the interior of the ventilator housing while a remainder of the clamping valve assembly remains attached within the interior of the ventilator housing; and
removing and replacing one or more of a first tube fluidly connecting the first inlet port and the first outlet port, and a second tube fluidly connecting the second inlet port and the second outlet port.

37. The method of claim 36, wherein:
one or more of the first inlet port, the first outlet port, the second inlet port, and the second outlet port are connected together as a first port block; and
removing and replacing at least one of the first inlet port, the first outlet port, the second inlet port, and the second outlet port comprises removing and replacing the first port block.

38. The method of claim 36, wherein:
the first inlet port and the second outlet port are connected together as a first port block;
the first outlet port and the second inlet port are connected together as a second port block; and
removing and replacing at least one of the first inlet port, the first outlet port, the second inlet port, and the second outlet port comprises removing and replacing the first port block and the second port block.

39. A service unit for a ventilator, the service unit comprising:
one or more sealed packages each comprising a respective sterile interior volume;
a first inlet port located in one of the respective sterile interior volumes;
a first outlet port located in one of the respective sterile interior volumes;
a second inlet port located in one of the respective sterile interior volumes;
a second outlet port located in one of the respective sterile interior volumes;
a first tube located in one of the respective sterile interior volumes; and
a second tube located in one of the respective sterile interior volumes.

40. The service unit of claim 39, wherein one or more of the first inlet port, the first outlet port, the second inlet port, and the second outlet port are connected together as a first port block.

41. The service unit of claim 40, wherein the first port block is configured to attach to a clamping valve assembly located in a ventilator housing without removing the clamping valve assembly from the ventilator housing.

42. The service unit of claim 39, wherein the first inlet port and the second outlet port are connected together as a first port block, and the first outlet port and the second inlet port are connected together as a second port block.

43. The service unit of claim 42, wherein the first port block and the second port block are configured to attach to a clamping valve assembly located in a ventilator housing without removing the clamping valve assembly from the ventilator housing.

44. The service unit of claim 43, wherein the first tube is fluidly connected between the first inlet port and the first outlet port and the second tube is fluidly connected between the second inlet port and the second outlet port in the sterile interior volume.

45. The service unit of claim 39, further comprising one or more air filters located in one of the respective sterile interior volumes.

46. A method for operating a ventilator clamping valve assembly comprising a clamp cradle having a first cradle surface and a second cradle surface, a clamp having a first clamp surface facing the first cradle surface to define a first volume between the first clamp surface and the first cradle surface and a second clamp surface facing the second cradle surface to define a second volume between the second clamp surface and the second cradle surface, a first tube extending through the first volume, and a second tube extending through the second volume, the method comprising:
(a) moving the first clamp surface towards the first cradle surface to simultaneously decrease the first volume to compress the first tube and increase the second volume to decompress the second tube;
(b) moving the second clamp surface towards the second cradle surface to simultaneously decrease the second volume to compress the second tube and increase the first volume to decompress the first tube; and
cyclically repeating steps (a) and (b) according to a respiration rate of a patient.

47. The method of claim 46, wherein:
moving the first clamp surface towards the first cradle surface comprises moving the clamp to a first clamp position in which the first tube is at least partially compressed between the first clamp surface and the first cradle surface and the second tube is at least partially compressed between the second clamp surface and the second cradle surface; and/or
moving the second clamp surface towards the second cradle surface comprises moving the clamp to a second clamp position in which the first tube is at least partially compressed between the first clamp surface and the first cradle surface and the second tube is at least partially compressed between the second clamp surface and the second cradle surface.

48. A ventilator valve assembly comprising:
a base;
a plurality of ports comprising: a first inlet port, a first outlet port, a second inlet port, and a second outlet port;
a first tube connecting the first inlet port and the first outlet port;
a second tube connecting the second inlet port and the second outlet port;
a first valve body configured to selectively change a cross-sectional area of the first tube;
a second valve body configured to selectively change a cross-sectional area of the second tube;
one or more motors connected to the first valve body and the second valve body and
control electronics configured to:
operate the one or more motors to simultaneously move the first valve body to reduce the cross-sectional area of the first tube and move the second valve body to increase the cross-sectional area of the second tube; and
operate the one or more motors to simultaneously move the first valve body to increase the cross-sectional area of the first tube and move the second valve body to decrease the cross-sectional area of the second tube.

49. The ventilator clamping valve assembly of claim 48, wherein the control electronics are further configured to:
operate the one or more motors to move the first valve body to change the cross-sectional area of the first tube and not move the second valve body to maintain the cross-sectional area of the second tube at a constant value; and/or
operate the one or more motors to simultaneously not move the first valve body to maintain the cross-sectional area of the first tube at a constant value and move the second valve body to change the cross-sectional area of the second tube.

50. The ventilator clamping valve assembly of claim 48, wherein the one or more motors comprises a single motor connected to move the first valve body and the second valve body in unison.

51. The ventilator clamping valve assembly of 50, wherein the first valve body and the second valve body are integrally formed.
